(19) United States
(12) Patent Application Publication
(10) Pub. No.: US 2003/0215858 A1
(43) Pub. Date: Nov. 20, 2003

(54) Enhanced gene expression system

(75) Inventor: Nancy Smyth Templeton, Houston, TX (US)

Correspondence Address: Elizabeth R. Hall, 1722 Maryland Street, Houston, TX, 77006-1718, US

(73) Assignee: BAYLOR COLLEGE OF MEDICINE, Houston, TX (US)

(21) Appl. No.: 10/406,510

(22) Filed: Apr. 3, 2003

Related U.S. Application Data
(60) Provisional application No. 60/370,900, filed on Apr. 8, 2002.
(60) Provisional application No. 60/430,780, filed on Dec. 4, 2002.

(51) Int. Cl. A61K 48/00; C12Q 1/68; C12N 15/00

(52) U.S. Cl. 435/6; 435/320.1; 514/44

(57) ABSTRACT

The present disclosure provides an enhanced gene transcription system including a systematic method of selecting efficient promoter-enhancers, of optimizing plasmid design and increasing transcription of a cDNA of interest in transfected target cells. The present invention identifies abundantly, selectively expressed genes and creates plasmids comprising the promoters-enhancers of those genes.

### CROSS REFERENCE TO RELATED APPLICATIONS

The present application, pursuant to 35 U.S.C. 111(b), claims the benefit of the filing date of provisional application Serial No. 60/370,900 filed Apr. 8, 2002, entitled “Enhanced Transcription for Robust Cancer Treatment” and of provisional application Serial No. 60/430,780 filed Dec. 4, 2002 entitled “Promoter Selection Process for Enhanced Transcription.”

### STATEMENT REGARDING FEDERALLY SPONSORED RESEARCH OR DEVELOPMENT

Research leading to this invention was federally supported, in part, by the Breast Cancer SPORE, National Institutes of Health, through Grant P50CA58183, and the U.S. Government has certain rights thereunder in this invention.

### BACKGROUND OF THE INVENTION

1. Field of the Invention

The present invention relates generally to the field of gene therapy. More particularly, the present invention relates to a system of enhanced transcription including a method of selecting tissue specific promoters.

2. Description of the Related Art

Gene therapy is being actively pursued as a means of treating a variety of diseases. The fundamental idea is to administer a functional gene, so as to give targeted cells a new protein-manufacturing capacity. This new functional gene would replace a gene that should have been present but is missing or defective in the treatment of monogenetic or single-gene mendelian diseases such as cystic fibrosis. In other situations a foreign gene, one that should not be functioning in that cell, might be introduced to a particular diseased cell and be used against that cell to kill it. This technique might become applicable especially to acquired illnesses, including heart disease and cancer. Because most monogenetic, inherited diseases are somewhat rare, gene therapy may have its greatest public-health impact in acquired illnesses.

In all cases, success is measured by how well an added gene functions to produce the therapeutic protein. The major problems lies in delivering the gene to the correct destination and in having it act with a useful duration to produce sufficient amounts of the desired therapeutic protein. Three important issues have become critical aspects that must be considered. First, the need for an effective delivery system to specifically target cells. Second, the vector titer or levels of plasmid DNA achieved in the target cell nucleus. Third, an effective gene expression system to produce the therapeutic protein.

Effective target cell targeting is inextricably linked to the titer of the vector that is transfected into the target cell. Ultimately, even when the titer of the vector in the transfected cell appears to be sufficient, efficient gene therapy can only be achieved when the plasmid is able to express the cDNA of interest at adequate levels in the target cell. In recent years much progress has been made in targeted delivery and investigators have reported high levels of plasmid DNA in the nucleus of transfected cells. However, despite high vector titers, the gene expression in these cells remained low or undetected. [Handumrongkul, et al.]

It is well established that the primary control of gene expression (i.e., the differences in the RNA and protein concentration of different tissues) is at the level of transcription. Transcription is the process of reading the genes, made up of a sequence of DNA, by the enzyme DNA dependent RNA Polymerase II (RNA Pol II). When the enzyme RNA Pol II reads the DNA it produces, directly from this template, another molecule called the heteronuclear RNA (hnRNA). The hnRNA is taken through a process within the nucleus where part of it, called the introns, are spliced out. This results in a mature messenger RNA (mRNA). The mRNA is then transported outside the nucleus of the cell to the cytoplasm where it is also read. From the reading of the template of the mRNA a protein is made.

It has been demonstrated that increased transcription of a particular type of gene, which ultimately produces a protein, is mediated by an increased rate of initiation of transcription by RNA Pol II. Enhancer and promoter recognition by RNA polymerase, transcription factors, and auxiliary proteins is a complex process thought to involve both primary and secondary sequence characteristics of the regulatory DNA. The initiation and mediation of transcription occurs at the region of the gene known as the promoter and may be further regulated by DNA sequences known as enhancers.

Promoters are nucleotide sequence elements within a nucleic acid fragment or gene, which controls the expression of that nucleic acid fragment or gene. Promoter sequences provide the recognition for RNA polymerase and other transcriptional factors required for efficient transcription. Promoters from a variety of sources can be used efficiently in eukaryotic cells and tissues to express sense and antisense gene constructs.

Enhancers are the nucleotide sequence elements, which can stimulate promoter activity. Enhancers respond to the signals mediated by the proteins regulating the transcription of the gene. Enhancers are regulatory nucleotide sequences that may be located next to or at a great distance (100's to 1000's of bps) upstream or downstream (5′ and/or 3′ end of the gene sequences) from the promoter that it influences. Enhancers can also be located within the introns. The regulative effect of the enhancers is either positive or negative. In the latter case they are generally called silencers. Enhancers can function in many different positions and in either orientation, and they can function when fused to a heterologous promoter. Eukaryotic genes typically have multiple enhancers, each with a special regulatory role (e.g., stimulates transcription in a particular temporal or spatial pattern or in response to a particular stimulus such as a steroid hormone). Enhancers are typically composed of clustered binding sites for multiple transcription factors.

The typical approach to selecting promoters for providing transcriptional levels adequate to meet a therapeutic need has been to use strong viral promoters with ubiquitous and constitutive activity. These promoters such as the cytomegalovirus promoter (CMV) or the simian virus 40 promoter-enhancer (SV40) have been the preferred expression control elements used in clinical trials; yet these promoters have proven to have deleterious effects because of their non-tissue specific nature and their highly unregulated gene expression. Furthermore, even allowing for the CMV or SV40 non-specific nature, the promoters have often proven insufficiently effective at inducing transcriptional levels adequate to meet therapeutic needs.

One approach to finding an appropriate promoter devoid of the non-tissue specificity of the CMV and SV40 promoters has been to investigate regulated promoters such as the mouse mammary tumor virus promoter-enhancer (MMTV) that provides steroid-regulated gene expression in murine cells. Unfortunately these promoters have not proven to be as tissue specific, or as robust as needed to meet therapeutically required expression levels.

Accordingly, there is a need in the art for a systematic method of selecting promoters and enhancers that can increase the efficacy of transcription in gene therapeutics.

### SUMMARY OF THE INVENTION

The present invention provides an enhanced gene transcription system and a systematic process for selecting efficient promoter-enhancers, of optimizing plasmid design and increasing transcription of a cDNA of interest in transfected target cells. The present invention identifies abundantly, selectively expressed genes and creates plasmids comprising the known or novel promoters-enhancers of those genes.

One aspect of the present invention is a process for selecting a promoter for inclusion in a plasmid used to transfect a target cell, the process comprising the steps of: (a) identifying a transcription product in high abundance in a target cell; (b) identifying a promoter associated with the transcription product; (c) inserting the promoter into a gene expression plasmid construct, the plasmid construct having a therapeutic gene to be expressed; (d) transfecting the target cell with the gene expression plasmid construct; and (e) verifying gene expression of the therapeutic gene in the target cell.

Another aspect of the present invention is a process for selecting a promoter for inclusion in a plasmid to be used in gene therapy comprising the steps of: (a) determining a gene expression level for a plurality of transcription products in a diseased tissue; (b) selecting a transcription product in high abundance in the diseased tissue and a target cell associated with the diseased tissue; (c) identifying a promoter or enhancer associated with the transcription product; (d) inserting the promoter or enhancer into a gene expression plasmid construct, the plasmid construct having a therapeutic gene to be expressed; (e) transfecting the target cell with the gene expression plasmid construct; and (f) verifying gene expression of the therapeutic gene in the target cell.

Yet another aspect of the present invention is a process for designing a plasmid for transfecting a target tissue, the process comprising the steps of: (a) selecting a gene expression plasmid having an origin of replication, a multiple cloning site, a therapeutic gene, a polyadenylation signal sequence, and an antibiotic resistant gene; (b) identifying a transcription product in high abundance in a cell line associated with a target tissue; (c) identifying a promoter or enhancer associated with the transcription product; (d) inserting the promoter into the gene expression plasmid in various locations close to the therapeutic gene to form a plurality of plasmid constructs; (e) transfecting the target cell line with each plasmid construct; (f) measuring gene expression of the therapeutic gene in the target cell line transfected with each plasmid construct; (g) selecting the plasmid constructs that provide efficient gene expression in the transfected target cell line; and (h) verifying gene expression of the selected plasmid constructs in the target tissue.

Still yet another aspect of the present invention is an expression plasmid comprising: an origin of replication gene; a polyadenylation site; an antibiotic resistant gene; a multiple cloning site; a therapeutic gene; and a promoter associated with a highly abundant transcription product of a target cell.

The foregoing has outlined rather broadly several aspects of the present invention in order that the detailed description of the invention that follows may be better understood. Additional features and advantages of the invention will be described hereinafter which form the subject of the claims of the invention. It should be appreciated by those skilled in the art that the conception and the specific embodiment disclosed might be readily utilized as a basis for modifying or redesigning the structures for carrying out the same purposes as the invention. It should be realized by those skilled in the art that such equivalent constructions do not depart from the spirit and scope of the invention as set forth in the appended claims.

### BRIEF DESCRIPTION OF THE DRAWINGS

For a more complete understanding of the present invention, and the advantages thereof, reference is now made to the following description taken in conjunction with the accompanying drawings, in which:

FIG. 1 is a schematic representation of the pVAX1 vector used as the backbone in the plasmid constructs;

FIG. 2 is a schematic representation of the p4119 vector, a portion of which was incorporated into most of the plasmid constructs;

FIG. 3 is a schematic of the p4119 vector shown in FIG. 2 specifically showing the portion of the vector inserted into the plasmid pCAT2;

FIG. 4 is a schematic of the plasmid pCAT2;

FIG. 5 is a schematic of the plasmid pCAT3;

FIG. 6 is a schematic of the p4119 vector shown in FIG. 2 specifically showing the portion of the vector inserted into the plasmid pCAT4;

FIG. 7 is a schematic of the plasmid pCAT4;

FIG. 8 is a schematic of the plasmid pCAT5;

FIG. 9 is a schematic of the plasmid pCAT6;

FIG. 10 is a schematic of the plasmid pCAT7;

FIG. 11 is a schematic of the p4119 vector shown in FIG. 2 specifically showing the portion of the vector inserted into the plasmid pCAT8;

FIG. 12 is a schematic of the plasmid pCAT8;

FIG. 13 is a schematic of the plasmid pCAT9;

FIG. 14 is a graphic representation of CAT expression in MCF7 cells transfected with the plasmid constructs pCAT-1 to pCAT-9;

FIG. 15A is a graphic comparison of pCAT-4 versus pCAT-8 expression in various breast cells lines;

FIG. 15B is a graphic comparison of pCAT-4 versus pCAT-8 expression in various lung cells lines;

FIG. 15C is a graphic representation of the fold increase in CAT expression in cells tranfected with pCAT-8 over the same cells transfected with pCAT-4;

FIG. 16A is a graphic comparison of pCAT-4 versus pCAT-8 expression in MCF7 cells grown in culture chambers containing 21%, 5.0%, or 9.9% oxygen post-transfection;

FIG. 16B is a graphic representation of the fold increase in the mean CAT expression of cells transfected with pCAT-4 or pCAT-8 when grown in 9.9% or 5.0% oxygen versus when grown in 21% oxygen;

FIG. 17A is a graphic comparison of CAT expression in MCF7 cells transfected with pCAT-4 and pCAT-8 when harvested at 24 hours, 7 days, or 14 days post-transfection;

FIG. 17B is a graphic representation of the fold increase in the mean CAT expression of MCF7 cells transfected with pCAT-8 versus pCAT-4 when harvested at 24 hours, 7 days or 14 days post-transfection;

FIG. 18A is a graphic comparison of pCAT-4 versus pCAT-8 expression in various breast tumors after intravenous injection or direct tumor injection of liposomal coated pCAT-4 and pCAT-8;

FIG. 18B is a graphic comparison of pCAT-4 versus pCAT-8 expression in the heart and lung of animals intravenously injected with liposomal coated pCAT-4 and pCAT-8;

FIG. 18C is a graphic representation of the fold increase in CAT expression in the tissues shown in FIGS. 18A and 18B that were tranfected with pCAT-8 over the same cells transfected with pCAT-4;

FIG. 19A is a graphic comparison of pCAT-4 versus pCAT-8 expression in various tissues of immune competent, normal mice after the intravenous injection of liposomal coated pCAT-4 and pCAT-8;

FIG. 19B is a graphic representation of the fold increase in the mean CAT expression in tissues from immune competent, normal mice injected intravenously with pCAT-8 versus pCAT-4; and

FIG. 20 is a schematic of a hypothetical plasmid construct with no viral sequences having a keratin-8 promoter-enhancer and a GAPDH promoter-enhancer.

### DESCRIPTION OF THE PREFERRED EMBODIMENTS

The implementation of gene therapy for acquired and inherited disorders requires improvements in many areas of the field, but most especially in the efficacy and stability of the regulation of the expression of the therapeutic gene within the specific cell targets. High transduction efficiency and titers are a basic necessity in order to get the therapeutic gene of interest into the specific cell population where the gene needs to be expressed. In many cases, even when the gene has been targeted and transferred with high efficiency to the correct cells the stability of the transgene expression has been difficult to achieve and/or the expression of the transgene does not attain the appropriate levels of protein product for therapeutic efficacy.

Cancers have a much more complex molecular biology than that of their normal cell counterparts because of the changes that take place at the level of the genome. Genes in tumors appear to be regulated differently than those same genes in normal cells (Bargou, R. C., et. al, 1997, Baldini N., 1997) and the use of a simple expression cassette under the control of a constitutive promoter is typically not useful. Gene therapy must be able to kill the specific cancer cells through the introduction of therapeutic proteins and to meet this criteria, strong viral promoters with usually ubiquitous and constitutive activity have been the preferred expression control elements used in clinical trials. These have proven to have deleterious effects because of their non-tissue specific nature and their highly unregulated gene expression.

Part of the inability to express the therapeutic transgene properly could be due to promoter non-recognition or inactivation by the targeted cells. Lack of efficacy in certain patients could be due to differences in the levels of transactivating factors (TAFs) in specific cancer or diseased cells. Enhancer and promoter recognition by RNA Pol II, transactivating factors, and auxiliary proteins is a complex process thought to involve both primary and secondary sequence characteristics of the regulatory DNA. The number, diversity, orientation, and placement of transactivating factor-binding sites within the transcription control region of cells are parameters that define gene expression.

The present invention includes a novel systematic approach to the selection of promoters-enhancers for gene therapy. This systematic approach identifies promoters and enhancers associated with abundantly transcribed proteins in specific cells and tissues.

A first step in this promoter selection process utilizes a quantitative measure of gene expression in different cells and tissues (e.g., the Serial Analyses of Gene Expression, microarray data, or other databases used to determine expression profiling) to identify mRNAs or proteins that are in high abundance in a specific target cell. Promoters and enhancers associated with those mRNAs or proteins would be recognized by the cellular transcriptional mechanisms of the cells and tissues and therefore efficiently transcribe mRNAs and/or highly translated proteins.

Thus, promoter-enhancer chimeras are selected that optimize gene expression in specific tissues and cells, including specific cancer cells. Promoters-enhancers are selected based upon data quantitatively measuring gene expression of mRNAs or different proteins in target cells or tissues. Serial Analyses of Gene Expression (SAGE) or microarray data from specific cells, tissues, or tumors identify highly expressed and specifically expressed proteins. Candidate promoters associated with such proteins are selected. For the treatment of tumors, candidate promoters are selected, but not limited to, ones for which the tumor transcriptional operation does not limit gene expression.

### EXAMPLE 1

### Methods for Determining Abundantly Selectively Expressed mRNAs or Proteins

A. SAGE (Serial Analysis of Gene Expression)

SAGE is based on a multistep procedure involving reverse transcription, restriction endonuclease-mediated digestion to produce oligonucleotides, ligations, and polymerase chain reaction (PCR). Briefly, SAGE converts polyadenylated mRNA into complementary DNA (cDNA) by reverse transcription. cDNAs are cut by restriction enzymes to produce oligonucleotide “tags” of 9-1 1 base pairs. The tags are then ligated together to form concatemers that are amplified by PCR and then subcloned and sequenced. The number of tags present indicates the prevalence of a specific mRNA (i.e., the higher the number of tags, the greater the prevalence of the message and gene product). For a novel gene, researchers query genomic databases with the sequence of the tag to determine the identity of the gene.

SAGE: Measuring Gene Expression

Serial Analysis of Gene Expression, or SAGE, is a technique designed to gain a quantitative measure of gene expression. The SAGE technique itself includes several steps utilizing molecular biological, DNA sequencing and bioinformatics techniques. These steps have been used to produce 9 to 11 base “tags”, which are then assigned gene descriptions. For experimental reasons, these tags are immediately adjacent to the 3′ end of the 3′-most NlaIII restriction site in cDNA sequences.

Online Data Analysis

Information on National Cancer Institute (NCI), National Institutes of Health (NIH) SAGE libraries and access to the data produced from these libraries can be reviewed and analyzed with several online tools via the xProfiler and Virtual Northern.

Tag to Gene Mapping

In order to support SAGE development, a comprehensive human SAGE tag to gene map has been produced. This process relies heavily on the National Center for Biotechnology Information's (NCBI's) UniGene project. UniGene is a project which groups similar GenBank DNA sequences into clusters, each of which is identified by a unique number or identifier. These unique identifiers are prefixed by two letters identifying a particular species (“Hs” indicates Homo sapiens, “Mm” Mus musculus, and “Rn” Rattus norvegicus). UniGene is useful to SAGE because, ideally, UniGene provides one identifier and description for a potentially large pool of similar GenBank sequences, and therefore provides a mechanism to “map” one tag to one or more UniGene clusters, and conversely, to identify the tag(s) which are mapped from a particular UniGene cluster. The Cancer Genome Anatomy Project, or CGAP, is an NCI-initiated and sponsored project that has begun to delineate the molecular fingerprint of the cancer cell. Many different chemical, molecular biological, sequencing and bioinformatics techniques are being utilized in this endeavor, including the generation of SAGE libraries and sequences (Lash, A. E., et al., 2000; and Lal, A., et al. 1999).

Serial Analysis of Gene Expression

Serial analysis of gene expression, or SAGE, is a technique designed to take advantage of high-throughput sequencing technology to obtain a quantitative profile of cellular gene expression. Essentially, the SAGE technique measures not the expression level of a gene, but quantifies a “tag” which represents the transcription product of a gene. A tag, for the purposes of SAGE, is a nucleotide sequence of a defined length, directly 3′-adjacent to the 3′-most restriction site for a particular restriction enzyme. As originally described (Velculescu, V E et al., 1995), the length of the tag was nine bases, and the restriction enzyme NlaIII. Current SAGE protocols produce a ten to fourteen base tag (Zhang L, et al., 1997), and, although NlaIII remains the most widely used restriction enzyme, enzyme substitutions are possible. The data product of the SAGE technique is a list of tags, with their corresponding count values, and thus is a digital representation of cellular gene expression.

SAGEmap Analysis Tools

The analysis tool for the pooling and comparison of SAGE library data, SAGEmap xProfiler has been designed for differential-type analyses. Similar libraries can be placed into one of two groups based on their characteristics (e.g., normal colon or colon cancer). Comparisons are then made between the two groups using a statistical test developed specifically for SAGE data by Stephen Altschul at NCBI. Two optional filtering functions are available for use, if desired. The first filter is based upon a commonly used statistical measure of dispersion, the coefficient of variance (100*s/M; s=std deviation, M=mean), and operates between values within a group (0%, default, inactivates). The second filter is based upon tag matches. A list of tags can be entered, and only results on those tags will be returned.

A virtual Northern tool, vNorthern, has been designed to accept a sequence as input. Possible tags are then extracted from this sequence, and links provided to access the data from the various SAGE libraries currently represented on the SAGEmap website.

SAGEtag to UniGene Mapping

The construction of a SAGE tag to gene mapping is a multistep, automated process. These steps comprise:

(a) separating out individual human sequences from GenBank submission records;

(b) assigning a SAGE tag to each sequence, by assigning sequence orientation through a combination of identification of a poly-adenylation signal (ATTAAA or AATAAA), a poly-adenylation tail, and a sequence label, and then extracting a 10 base tag 3′-adjacent to the 3′-most NlaIII site (CATG) using information from NCBI's UniGene project; and

(c) assigning a UniGene identifier to each human sequence with a SAGE tag.

The result of this process is a SAGE tag to UniGene identifier mapping, with forward and reverse sequence frequency weights (w) given to the connections.

Resultant Mappings

Two tag to gene mappings result from this entire process. One is a “full” mapping, and the other a “reliable” mapping. Both of these are provided on the SAGEmap FTP site as downloadable files (very large), as well as integrated with the SAGE library data on this site through a searchable interface. If one wishes to search for a small number of tags, the searchable interface can be used on the Tag to Gene and Gene to Tag pages. The reliable mapping is also used in the display of tabulated SAGE tag data from the various SAGE libraries.

B. Microarray Analysis

The goal of comparative cDNA hybridization is to compare gene transcription in two or more different kinds of cells. Cells from two different tissues (i.e., cardiac muscle and prostate epithelium) are specialized for performing different functions in an organism. Although cells can be recognized from different tissues by their phenotypes, it is the regulation of the expression of the genes that makes one cell function as smooth muscle, another as a neuron, and still another as prostate. Ultimately, a cell's role is determined by the proteins it produces, which in turn depend on its expressed genes. Comparative hybridization experiments can reveal genes which are preferentially expressed in specific tissues. Some of these genes implement the behaviors that distinguish the cell's tissue type, while other controlling genes make sure that the cell only performs the functions for its type.

C. Usefulness of Measuring Expression Changes

Genetic disease is often caused by genes that are inappropriately transcribed—either too much or too little—or which are missing altogether. Such defects are especially common in cancers, which can occur when regulatory genes are deleted, inactivated, or become constitutively active. Unlike some genetic diseases (e.g. cystic fibrosis) in which a single defective gene is always responsible, cancers which appear clinically similar can be genetically heterogeneous. For example, prostate cancer (prostatic adenocarcinoma) may be caused by several different, independent regulatory gene defects even in a single patient. In a group of prostate cancer patients, every one may have a different set of missing or damaged genes, with differing implications for prognosis and treatment of the disease.

Quantitatively measuring the expression of genes in specific tissues under specific environments can serve two purposes in studying disease. It can pinpoint the transcription differences responsible for the change from normal to disease cells, and it can distinguish different patterns of abnormal transcription in heterogeneous diseases such as cancer. Cancers are common examples of genetically heterogeneous diseases, but they are by no means the only ones. Patients with diabetes, heart disease, and multiple sclerosis have diseases for which genetic risk factors are known to be heterogeneous.

Expression changes of interest also include the introduction of signaling molecules, such as hormones, interleukins, and interferons, as well as the actions of drugs into a cell model system or a patient biopsy's tissue in culture. All these molecules stimulate a change in a cell's behavior (including possibly its death). While some of the changes may be mediated purely at the protein level, others require new transcription which can be detected by the quantitative measure of gene expression in a cell.

During cancer growth cells undergo DNA replication, mitosis, and eventually death. These activities require quite different gene products, such as DNA polymerases for genome replication or microtubule spindle proteins for mitosis. A cell's genes encode the “programs” for these activities, and gene transcription is required to execute those programs. Quantitatively measuring the expression of genes at different times in the cell cycle can assist in identifying pathways responsible for controlling cell growth.

Thus, quantitatively measuring the expression of genes in specific tissues in healthy and disease states can be used to extrapolate the cellular transcriptional mechanisms available in those tissues, and the variations in those mechanisms that occur in specific disease states. This allows the selection of promoter-enhancer chimeras, pursuant to the present invention, for use in gene therapy in those tissues.

### EXAMPLE 2

### Determining Abundantly, Selectively Expressed Proteins

The SAGE databases and associated tools are available on-line and provide an excellent mean of finding the quantity of mRNA for every expressed known or unknown gene (Velculescu, V. E., et al., 1995). To identify more efficient promoters-enhancers for specific cell lines or tissues, the SAGE databases were searched for highly expressed genes in a designated tissue (breast) and cell libraries versus other tissues libraries (e.g., brain, colon, endothelium, and prostate). The SAGEmap xProfiler is employed for determining the transcription of genes in cancer cells versus normal cells. SAGE map xProfiler is found at http://www.ncbi.nlm.nih.gov/SAGE/sagexpsetup.cgi. Table 1 lists several highly abundant and selectively expressed transcripts identified by this analysis.

SAGE Virtual Northern analysis was used to provide sequence data across different tissue and cell types [see http://www.ncbi.nlm.nih.gov/SAGE/sagevn.cgi]. The SAGE gene to tag mapping provided a method to query expression levels of specific genes in the entire database [see http://www.ncbi.nlm.nih.gov/SAGE/SAGEcid.cgi].

The NCI, NIH SAGE databases that were analyzed included both breast tumor cell lines (e.g., MCF7, HMEC-B41, MDA-453, SK-BR-3, and DCIS2-purified cells) and microdissected human cancers (e.g., DCIS malignant breast tissue) and normal tissues (e.g., mammary epithelium and luminar epithelial cells). Both tissue and cell SAGE libraries were screened to determine which genes are highly expressed at the mRNA level in both the tumor cell lines used in the xenograft models being studied and in authentic human breast tumors. This analysis thus identified genes whose promoters should be useful both for animal studies and for therapeutic application in clinical trials for breast cancer.

The SAGE data for deoxythymidylate kinase, N-Ras related protein, keratin-8, ribosomal L30, glycerealdehyde 3-phosphate dehydrogenase (GAPDH), and interferon alpha (IFNa)-inducible protein gene expression in various cell lines and tissues is shown in Table 1.

TABLE 1
Gene Expression Levels
(SAGE tag frequency/10⁶)

| | UniGene ID Hs: | | | | | |
|---|---|---|---|---|---|---|
| | 79006 | 69855 | 242463 | 111222 | 169476 | 265827 |
| | deoxy-thymidylate | N-Ras related | keratin-8 | Ribosomal L30 | GAPDH | IFN-Inducible |
| MCF7 Control O hr. | 9381 | 6267 | 12622 | 9381 | 6958 | 3962 |
| MCF7 + Estrogen 10 hr. | 9630 | 5807 | 12492 | 9630 | 4351 | 2945 |
| Mammary Epithelium | 1342 | 101 | 1261 | 1342 | 549 | 40 |
| DCIS | 3783 | 0 | 800 | 3783 | 339 | 194 |
| HMEC-B41 | 3546 | 0 | 1418 | 3546 | 6382 | 0 |
| MDA-453 | 1743 | 105 | 2536 | 1743 | 951 | 158 |
| SK-BR-3 | 4660 | 490 | 6623 | 4660 | 1962 | 0 |
| DCIS2-Purified Cells | 2215 | 173 | 1523 | 2215 | 450 | 7338 |
| BrN-Normal Luminar | 2582 | 79 | 718 | 2582 | 532 | 0 |
| Epithelial Cells | | | | | | |

The N-Ras related protein and IFNa-inducible protein genes were inefficiently transcribed in breast tissue (both normal breast and breast tumor tissue) and certain breast cancer cell lines. Thus, promoters for these proteins were discarded from consideration for use in gene therapy for breast cancer. In contrast, promoters for deoxythymidylate kinase, keratin-8, ribosomal L30, and glycerealdehyde 3-phosphate dehydrogenase (GAPDH) were considered candidate promoters for efficient expression of transgenes in breast cancer cells as well as normal breast. The GAPDH gene transcripts were highly abundant in breast cancer cells, particularly in MCF7 cells, and were underrepresented in normal mammary epithelium (see Table 1).

The SAGE data indicated that the human glyceraldehyde 3-phosphate dehydrogenase (GAPDH) gene promoter is a promoter-enhancer that is widely recognized by TAFs in several breast cancer cells and not in normal breast tissue. The GAPDH gene promoter sequence has been characterized (Aki, T., et al. 1997) and is sold commercially (InvivoGen, San Diego, Calif.). GAPDH protein is also up-regulated during hypoxia (Escoubet, B, et. al., 1999 and Graven, K K, et. al., 1994), as is common in tumors therefore the HIF-1 DNA binding site (Graven K K, et. al., 1999) may play a very strong role in the hypoxic response.

The SAGE data suggests also that the keratin-8 promoter would be useful for efficient expression of transgenes in breast cancers, and not in normal breast. Promoters from two other keratins, 14 and 19, have been used extensively to target transgene expression to other tissues in mice (Del Rio, M, et al., 1999, Sinha, S, 2000, Sinha, S, 2001, Brembeck, F H, 2001, and Waikel, R L, et. al., 2001). Thus, according to the present invention the keratin-8 promoter, as identified by xProfiler, is an excellent candidate for tissue- and/or disease-specific plasmids with or without known viral promoters.

### EXAMPLE 3

### Construction of Plasmids

Therapeutic and reporter gene expression were examined within certain cell lines after non-viral plasmid DNA delivery. Three p53 containing plasmids were constructed to test the efficacy of the pVAX1 and the 4119 vector components in the transcription of the p53 in HCC 1427, a p53 null breast cancer cell line. In addition, nine different plasmids were constructed with a reporter gene and used to test their in vitro and in vivo transcription efficiency.

Chloramphenicol acetyltransferase protein is widely used as a reporter gene because its concentration within cells is easy to measure and its gene is not eukaryotic, therefore its expression in mammalian cells is solely from the inserted vector. It is an enzyme that inactivates the antibiotic chloramphenicol from Streptomycetes venezuelae by acetylation.

The p53 gene was used in these experiments because it has been used in several cancer clinical trials, albeit resulting in both successes and failures. It is a transactivating factor that is capable of activating a variety of genes involved in cell cycle arrest and is able to repress gene expression/function. The consequence of gene activation and/or repression is growth arrest and/or apoptosis. However, mutations in p53 can occur, resulting in functional defects that allow the cancerous cell to evade growth arrest and apoptotic signals.

The majority of p53 mutations in human cancer are thought to be dominant-negative. This means that mutant allele products that have lost wild-type p53 function also have a deleterious effect on the function of the wild-type allele product in transcriptional regulation, growth arrest, and apoptosis. These differences also compromise the efficacy of apoptosis-inducing drugs that require TAFs in synergy to propagate specific signal transduction pathways. Inserting a wild-type p53 gene into cells that lack this function can restore wild-type p53 function. However, p53 functions as a tetramer with regard to its transcriptional activity so it is important to express high levels to ensure sufficient quantities of functional protein. When the cells express a mutant p53 and a wild-type peptide heterodimerase in the same cell, the mutant peptide can abrogate the function of the wild-type peptide expressed from the transfected gene. This problem of dominant-negative mutants, preventing restoration of wild-type function has serious consequences for future gene therapy. Therefore, it is important to obtain maximum p53 expression in cancer cells.

Large differences have been observed in the level of gene expression among individual tumors which suggested that differences between the transactivating factors (TAF) within the cancer cells that recognized their specific promoter-enhancer constructs were responsible for the different levels in gene expression. Because tumors within a patient or among patients are heterogeneous, this heterogeneity can account for the differences in gene expression observed within clinical trials. The variations in gene expression shown in the experiments discussed below are not explained by differences in delivery of plasmid DNA to the nuclei of high and low expressing cells, as transfection studies using fluorescent-labeled plasmid DNA showed no differences among levels of DNA in the nuclei (data not shown).

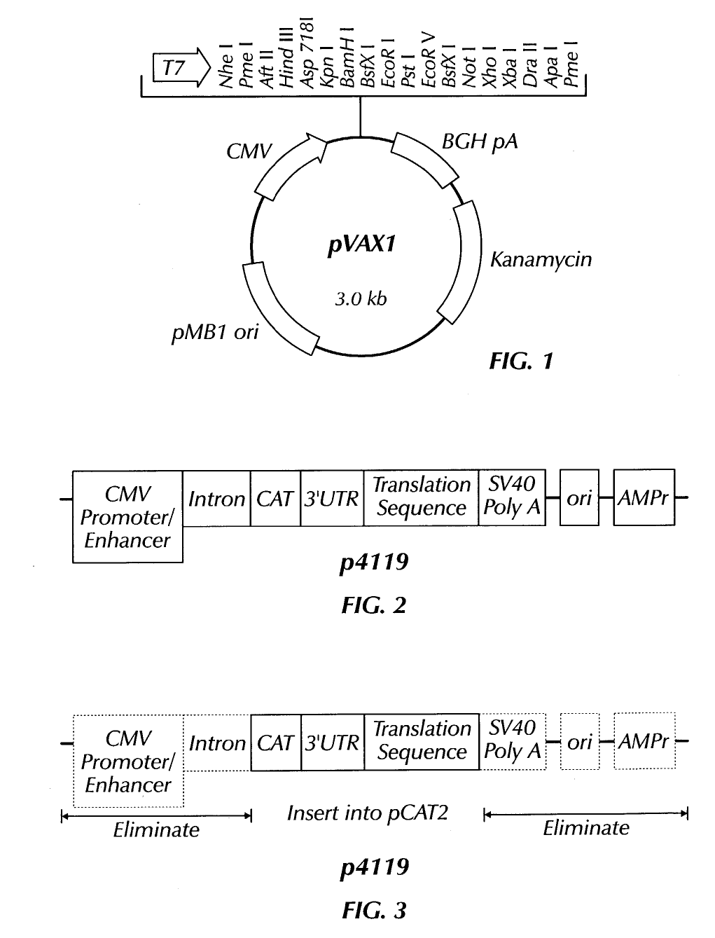

Several different chimeric promoters were constructed using the pVAX1 plasmid purchased from Invitrogen, Carlsbad, Calif., shown in FIG. 1, and the p4119 plasmid, shown in FIG. 2, that was kindly provided by Dr. Robert Debs from California Pacific Medical Center Research Institute, San Francisco, Calif. The human GAPDH promoter-enhancer was obtained from the pDRIVE-hGAPDH plasmid purchased from Invivogen, San Diego, Calif.

The pVAX1 is the backbone vector used to form the chimeric promoters. The pVAX1 was commercially obtained from Invitrogen, Carlsbad, Calif. The pVAX1 is engineered to be a very simplified, streamlined vector. The pVAX1 contains a minimal E. coli origin of replication constructed to limit vector size, but to have the same activity as the longer Ori (pMB1). The pVAX1 vector also contains a “sub-optimal” cytomegalovirus (CMV) promoter-enhancer, a T7 promoter/priming site, a multiple cloning site (bases 696-811), a bovine growth hormone polyadenylation signal sequence (BGH pA), and a kanamycin antibiotic resistant gene. The CMV promoter-enhancer within this vector drives the expression of the kanamycin resistance gene, which is used for the detection of cells expressing that protein.

As shown in FIG. 2, the p4119 vector contains a complete “optimal” CMV promoter/enhancer, an intron, the CAT reporter gene, a 3′ untranslated region (UTR), a translation sequence for optimal translation of the gene, a simian virus 40 poly adenylation signal sequence (SV40 poly A), a bacterial origin site of replication (ori) and an ampicillin resistance gene sequence (AMPr).

The p53 expressing plasmids were constructed as follows: the p53-1 is similar to the 4119 vector, but it contains the p53 gene instead of the CAT gene; the p53-2 plasmid was pVAX1 based, but contains the 3′UTR and the Translation Sequence of the p4119 vector; and the p53-3 plasmid contains the pMB1 ori and the Kanamycin gene of the pVAX1 and the CMV promoter-enhancer, intron, 3′UTR, Translation Sequence and SV40 Poly A genes from the p4119 vector.

HCC 1428 cells were tested after the non-viral delivery of the p53 expressing plasmids (i.e., p53-1, p53-2 and p53-3). The presence of p53 was assessed using quantitative Western blot analyses. The results showed that only the p53-3 produced significant levels of the p53 gene expression in the breast cancer cells. However, similar experiments in transfected H1299 lung cancer cells showed as much or more p53 being produced in lung cancer cells. The inefficient and non-specific transcription of these cells in response to the CMV promoter-enhancer of the p53 expressing plasmids led to the testing of plasmids containing the CAT reporter gene as described below.

The GAPDH promoter-hypoxia enhancer identified in the SAGE screening analysis was obtained commercially from InvivoGen, San Diego, Calif. and used in the plasmid constructs. The GAPDH gene encodes a key regulatory enzyme of glycolysis and has been commonly considered a constitutive housekeeping gene. However, the SAGE databases show different levels of GAPDH transcripts in breast tumor versus normal cells (Table 1). The inclusion of the hypoxia enhancer, suggested by the SAGE screening analysis, is consistent with the use of hypoxia responsive elements to increase gene expression within tumors (Cao, Y. J., et al. 2001; Ruan, H., et al. 2001; Ido, A., et al. 2001; Dachs, G. U., et al. 2000; Modlich, U., et al. 2000; Shibata, T., et al. 2000).

The chloramphenicol acetyltransferase (CAT) expressing plasmids were constructed as follows:

pCAT-p4119.

This plasmid is equal to the 4119 vector containing the CAT gene (Zhu, N, et. al., 1993).

pCAT-1.

This plasmid has the Elongation Factor 1-alpha (EF-1α) promoter-enhancer, the CAT gene and the pEFIRES backbone. The pEFIRES plasmid was obtained from Ming Zhang at the Baylor College of Medicine, Houston, Tex. The EF-1α is one of the most abundant transactivating proteins in eukaryotic cells and is expressed in almost all mammalian cells.

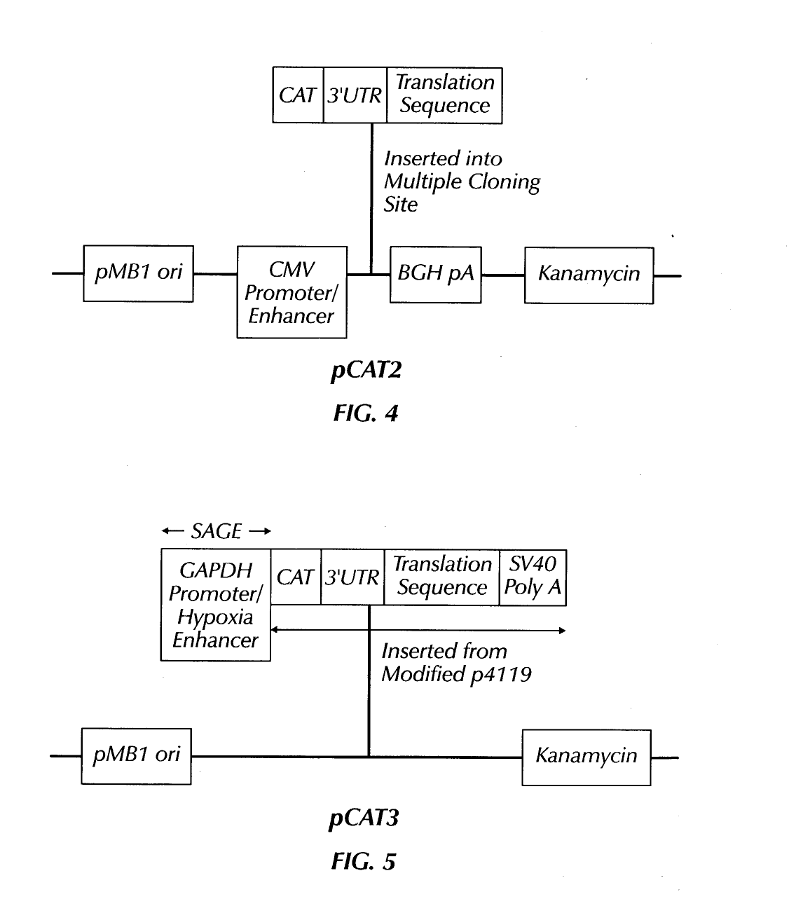

pCAT-2 (Shown in FIG. 4).

The pCAT-2 vector is pVAX1 based. However, as shown in FIGS. 3 and 4, the p4119 vector was used to contribute the CAT gene, 3′ UTR, and the Translation Sequence to pCAT-2. These sequences were excised from the p4119 vector and annealed into the multiple cloning site of the pVAX1 vector. Thus the pCAT-2 vector contains a sub-optimal CMV promoter-enhancer from the pVAX1 vector and a minimized amount of bacterial sequences.

pCAT-3 (Shown in FIG. 5).

The pVAX1 vector was used to contribute the pMB1 ori and the Kanamycin gene to the pCAT-3. The CMV promoter-enhancer, the intron, the ori and the AMPr genes were eliminated from the p4119 vector and the remaining sequences were inserted into the pVAX1 between the pMB1ori and the Kanamycin genes. The GAPDH promoter-hypoxia enhancer, also called the GAPDH promoter-enhancer, was inserted into the pCAT-3 vector upstream of the CAT gene. The GAPDH promoter-enhancer was the only promoter-enhancer in the pCAT-3.

pCAT-4 (Shown in FIG. 7).

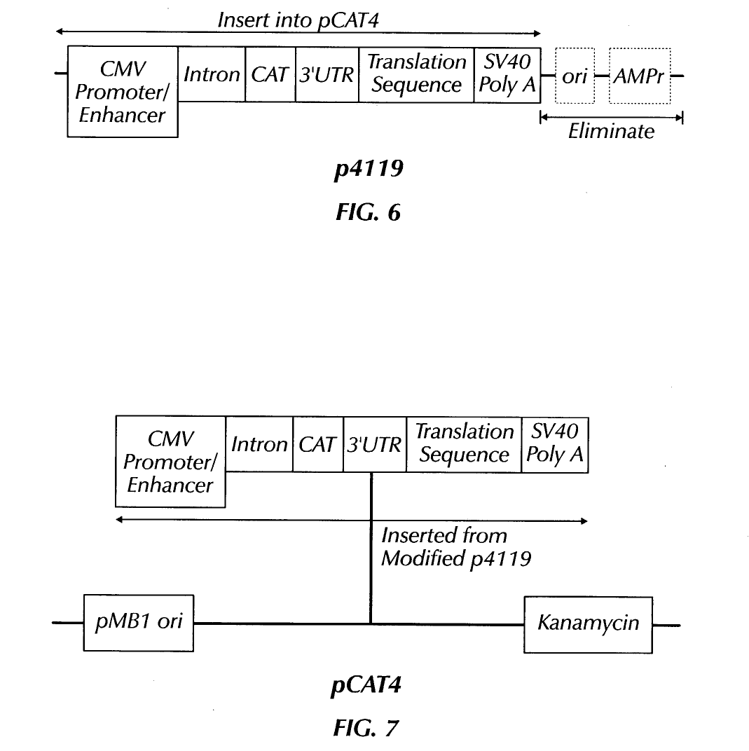

The pVAX1 vector was used to contribute the pMB1 ori and the Kanamycin gene to the pCAT-4. The ori and the AMPr genes were eliminated from the p4119 vector, as shown in FIG. 6, and the remaining sequences were inserted into the pVAX1 between the pMB1ori and the Kanamycin genes. The pCAT-4 vector contains an optimal CMV promoter-enhancer from the p4119 vector, plus a minimized amount of bacterial sequences from pVAX1. The pCAT-4 vector also contains an intron and more bacterial sequences from the p4119 vector than the pCAT-2 and p53-2 vectors. Extra bacterial sequences can have toxic effects on eukaryotic cells and are viewed as sub-optimal.

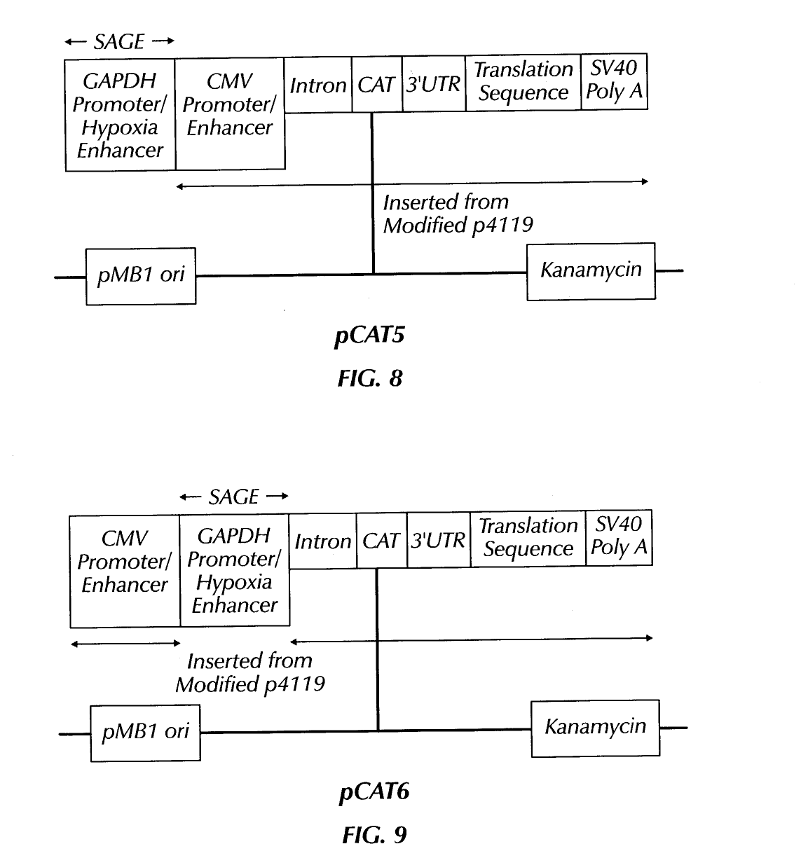

pCAT-5 (Shown in FIG. 8).

The pCAT-5 was created like the pCAT-4, except that the GAPDH promoter-hypoxia enhancer was inserted upstream of the CMV enhancer-promoter.

Further plasmid engineering was done to determine the optimal location for the GAPDH promoter-hypoxia enhancer and other GAPDH regulatory elements and to remove excess bacterial sequences.

pCAT-6 (Shown in FIG. 9).

The pCAT-6 was created by modifying pCAT-5 by reversing the order of the CMV enhancer-promoter and the GAPDH promoter-hypoxia enhancer (compare FIG. 8 and FIG. 9).

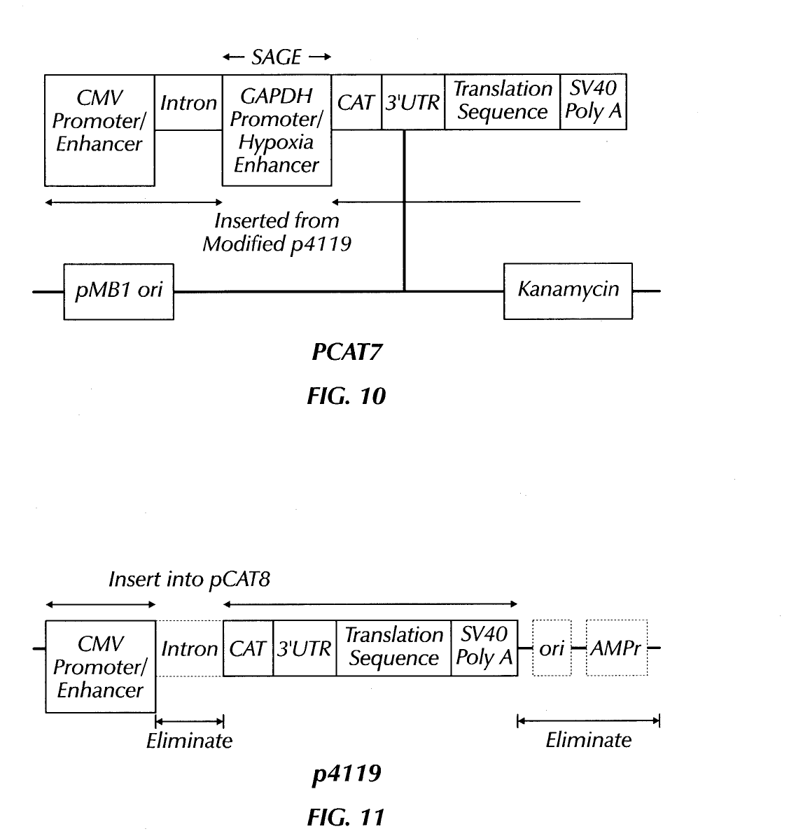

pCAT-7 (Shown in FIG. 10).

The pCAT-7 is similar to pCAT-6 except that the p4119 intron is placed between the CMV enhancer-promoter and the GAPDH promoter-hypoxia enhancer, rather than after the GAPDH promoter-enhancer (compare FIG. 9 and FIG. 10).

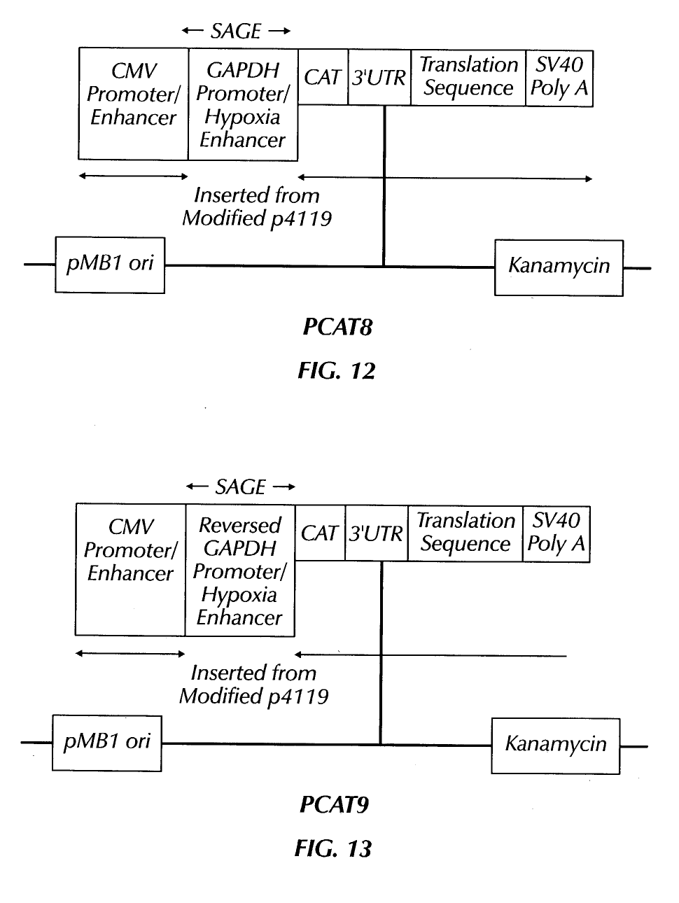

pCAT-8 (Shown in FIG. 12).

The pCAT-8 was created by modifying pCAT-7 by removing the intron (compare FIG. 10 and FIG. 12).

pCAT-9 (Shown in FIG. 13).

The pCAT-9 is constructed exactly the same as pCAT-8 except the GAPDH promoter-hypoxia enhancer sequence has been placed in the reverse direction.

Plasmid Preparation

The pEFIRES plasmid was obtained from Ming Zhang at the Baylor College of Medicine, and the p4119 plasmid was obtained from Robert Debs. The pVAX1 plasmid was purchased from Invitrogen, Carlsbad, Calif. The human GAPDH promoter-hypoxia enhancer was obtained from the pDRIVE-hGAPDH plasmid purchased from InvivoGen, San Diego, Calif. Plasmid design and construction is described above. All plasmids were grown under kanamycin selection in DH5α E. coli, with the exception of the pEFIRES-based plasmid, the pCAT-1, that were grown under ampicillin selection. All plasmids were purified by anion exchange chromatography using the Qiagen Endo-Free Plasmid Giga Kit, Qiagen, Germany. All plasmid pellets were resuspended in 10 mM Tris-HCl pH 8.0 and stored at −20° C.

### EXAMPLE 4

### Comparison of Novel Plasmids by In Vitro Transfection of MCF7 Cells

The nine plasmids designated pCAT-1 through pCAT-9 were used for transfections with extruded DOTAP liposomes prepared by a protocol previously developed and reported (Templeton, N. S., et al. 1997). These liposomes transfect a wide variety of cells in vitro (Yotnda, P., et al. 2002; Templeton unpublished data).

A. Methodology

C. Kent Osborne (Baylor College of Medicine, Houston, Tex.) provided the MCF7 cell line. Cells were cultured in 10% fetal calf serum and 10-12 μM insulin (Gibco/BRL, Gaithersburg, Md.). Cell lines were cultured in 6-well tissue culture clusters to 70% confluency.

DNA-liposome complexes were prepared as previously described (Templeton, N. S., et al. 1997). However, synthetic cholesterol (Sigma, St. Louis, Mo.) was substituted for cholesterol purchased from Avanti Polar Lipids (alabaster, Ala.) and used at 50:45 DOTAP:Chol. The liposomes used are bimellar invaginated vesicles.

Cells were transfected with extruded DOTAP DNA:liposome complexes using 5 μg of DNA per well. Transfections were performed in serum-free medium for three hours. Six independent in vitro transfections were performed for each data point reported.

Enzyme-linked immunosorbent assays (ELISAs) were performed using the Roche (Indianapolis, Ind.) CAT ELISA kit. Three control wells for each cell line were transfected with liposomes alone to determine any background levels of CAT production. All CAT protein determinations were corrected for any CAT immunoreactivity detected in the control cells. Protein determinations were performed using the Micro BCA kit (Pierce, Rockford, Ill.). The data is reported as the mean±S.D. Two-sided Student's t-tests were used to determine the p-values reported.

B. Comparative In Vitro Transfection

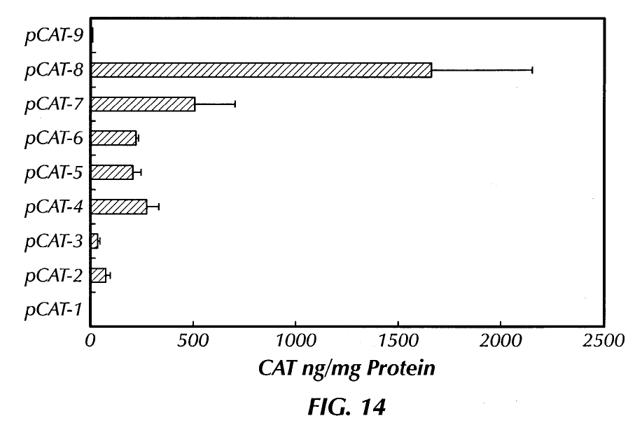

Using the pEFIRES plasmid that contains only the elongation factor-1α (EF-1α) promoter-enhancer to express the CAT gene, pCAT-1 was constructed. In previous work, a pEFIRES-maspin cDNA construct was encapsulated in the extruded DOTAP:Chol liposomes and demonstrated efficacy in a syngeneic breast tumor mestastasis model after intravenous or direct tumor injections (Shi, H. Y., et al., 2002). The mammary gland tumors were established using a PyV MT parental tumor cell line that was isolated from MMTV-polyoma virus Middle T transgenic mice. However, pCAT-1 produced no detectable levels of CAT production after transfection in MCF7 cells, as shown in FIG. 14.

The pVAX1 plasmid (Invitrogen, Carlsbad, Calif.) contains a short CMV promoter-enhancer of approximately 600 base pairs (bp). Even though the CMV promoter-enhancer of the pVAX1 is sub optimal, this plasmid is useful because the pVAX1 backbone is minimized for bacterial sequences and contains a kanamycin resistance gene to use for antibiotic selection during plasmid growth. For plasmid DNA used for human clinical trials, kanamycin selection is used and ampicillin selection is prohibited.

The CAT-2 plasmid contains the CAT gene subcloned into pVAX1. As shown in FIG. 14, the CAT-2 plasmid produced low levels of CAT after transfection in MCF7 cells. The pVAX1 CMV promoter-enhancer was removed from pCAT-2 and replaced with the GAPDH promoter-hypoxia enhancer to produce pCAT-3. CAT production in MCF7 cells transfected with CAT-3 also produced low levels of CAT as illustrated in FIG. 14.

The p4119 vector is a CAT plasmid designed for in vivo gene delivery and gene expression (Zhu, N., et al. 1993; Liu, Y., et al. 1995). This plasmid contains a longer length CMV promoter-enhancer of approximately 800 bp and an intron of 400 bp 5′ to the start codon of the CAT gene. However, the p4119 plasmid backbone is longer than that in pVAX1, containing about 590 bp more bacterial sequences, and contains the ampicillin resistance gene for selection, rather than the preferred kanamycin. The pCAT-4 construct was prepared by replacing the pVAX1 CMV promoter-enhancer in pCAT-2 with the p4119 CMV promoter-enhancer and intron. As shown in FIG. 14, the pCAT-4 transfected MCF7 cells produced a 3.7-fold increased CAT production compared to pCAT-2 transfected MCF7 cells (p<0.01).

The plasmids pCAT-5, -6, and -7 are modified from pCAT-4 by inserting the GAPDH promoter-hypoxia enhancer in different locations. The pCAT-5 contains the GAPDH promoter-hypoxia enhancer 5′ to the p4119 CMV promoter-enhancer. The pCAT-6 contains the GAPDH promoter-hypoxia enhancer 3′ to the p4119 CMV promoter-enhancer and 5′ to the p4119 intron. The pCAT-7 contains the GAPDH promoter-hypoxia enhancer 5′ to the CAT gene. After transfection of MCF7 cells, only the pCAT-7 produced a 1.9-fold increased CAT production compared to pCAT-4 (p<0.025) and a 7-fold increased CAT production compared to pCAT-2 (p<0.025). These results, illustrated in FIG. 14, suggest that it is better not to have an intron between the promoter-enhancer and the CAT gene.

To improve pCAT-7, the p4119 intron was removed to bring the p4119 CMV promoter-enhancer closer to the GAPDH promoter-hypoxia enhancer. This construct was named pCAT-8 and produced the highest levels of CAT production after transfection of MCF7 cells (see FIG. 14) at 1656.2 ng of CAT per mg total protein (ng CAT/mg protein). Therefore, pCAT-8 produced a 6.2-fold increased CAT production compared to pCAT-4 (p<0.05) and a 22.5-fold increased CAT production compared to pCAT-2 (p<0.05).

By reversing the GAPDH promoter-hypoxia enhancer sequences within pCAT-8 to create pCAT-9, CAT production dropped to nearly an undetectable level of CAT production (i.e., 3.3 ng of CAT/mg protein) after transfection of MCF7 cells. These data suggest that the highest level of increased gene expression and protein production in MCF7 cells were mediated by the GAPDH promoter-hypoxia enhancer sequences within the plasmid.

Other plasmids were tested that contained deletions, with or without the hypoxia enhancer sequences, in the GAPDH promoter-hypoxia enhancer region within pCAT-8. The plasmid constructs without the hypoxia enhancer sequences all produced reduced levels of CAT- expression compared to the pCAT-8 (data not shown) showing the advantage of including a hypoxia enhancer.

### EXAMPLE 5

### In Vitro Transfection of Various Lung Cancer and Breast Cancer Cells

To determine any cell-type specificity of increased CAT production mediated by the GAPDH promoter-enhancer sequences, a variety of different breast cancer and lung cancer cells were transfected with a plasmid containing the GAPDH promoter-enhancer and one without the GAPDH promoter-enhancer.

A. Cell Lines

The MCF7 cell line used was provided by C. Kent Osborne (Baylor College of Medicine, Houston, Tex.). Jack A. Roth (M. D. Anderson Cancer Center, Houston, Tex.) provided the H358, H460 and H1299 cell lines. The T-47D, SK-BR-3, A549, and HCC1428 cell lines were purchased from the American Type Culture Collection (ATCC), Manassas, Va. HCC1428 was recently deposited into the ATCC and was submitted by Adi F. Gazdar (University of Texas Southwestern Medical Center, Dallas, Tex.). HCC1428 is a p53 null human ductal breast carcinoma cell line with low levels of HER2/neu (14).

B. Methodology

Cell lines were cultured in 6-well tissue culture clusters to 70% confluency. DNA-liposome complexes were prepared as described above. Cells were transfected with extruded DOTAP DNA:liposome complexes using 5 μg of DNA per well. Transfections were performed in serum-free medium for three hours. Six independent in vitro transfections were performed for each data point reported.

Enzyme-linked immunosorbent assays (ELISAs) were performed using the Roche (Indianapolis, Ind.) CAT ELISA kit. Three control wells for each cell line were transfected with liposomes alone to determine any background levels of CAT production. All CAT protein determinations were corrected for any CAT immunoreactivity detected in the control cells. Protein determinations were performed using the Micro BCA kit (Pierce, Rockford, Ill.).

C. Comparative Cell Transfection

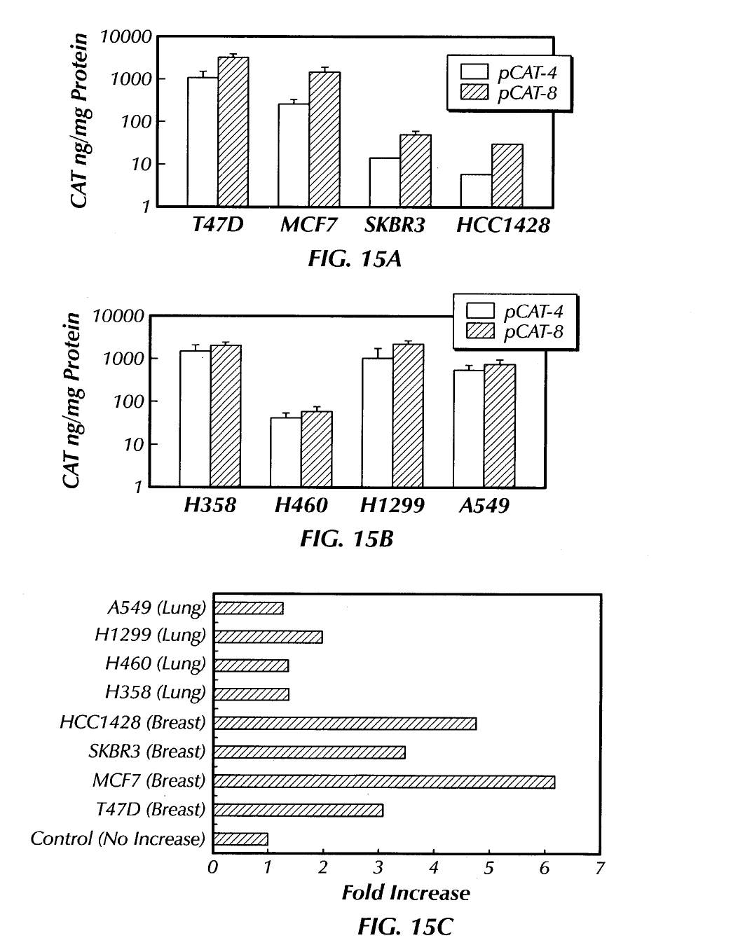

A variety of different breast cancer and lung cancer cells were transfected with the pCAT-4 plasmid that does not contain the GAPDH promoter-enhancer and the pCAT-8 plasmid containing the GAPDH promoter-enhancer that produced the highest levels of CAT production in our initial experiments (see FIG. 14). The breast cancer cell lines transfected were T-47D, MCF7, SK-BR-3, and HCC1428 (see FIG. 15A). The lung cancer cell lines transfected were H358, H460, H1299, and A549 (see FIG. 15B).

FIG. 15C shows the fold-increased CAT production in each cell line after transfection with pCAT-8 versus pCAT-4. The control is 1-fold and indicates no increase. The pCAT-8 increased CAT production in all breast cancer cells between 3.1 to 6.2-fold. Whereas, the pCAT-8 increased CAT production in all lung cancer cells between 1.3 to 2-fold. These data show that the GAPDH promoter-enhancer improved CAT production after transfection significantly higher in breast cancer cells than in lung cancer cells.

### EXAMPLE 6

### Comparison of CAT Production in MCF7 Cells Grown in Reduced Levels of Oxygen

To assess increased levels of CAT production by pCAT-8 contributed by the hypoxia enhancer within the GAPDH sequences, MCF7 cells were transfected with pCAT-4 and pCAT-8 and cultured in the standard (21%) or reduced (5.0% or 9.9%) levels of oxygen post-transfection. Oxygen levels in tumors have been measured, and tumor hypoxia exists at 1.3% and lower levels of oxygen [Vaupel, 1996]. Whereas normal oxygenated tissue has about 5% oxygen.

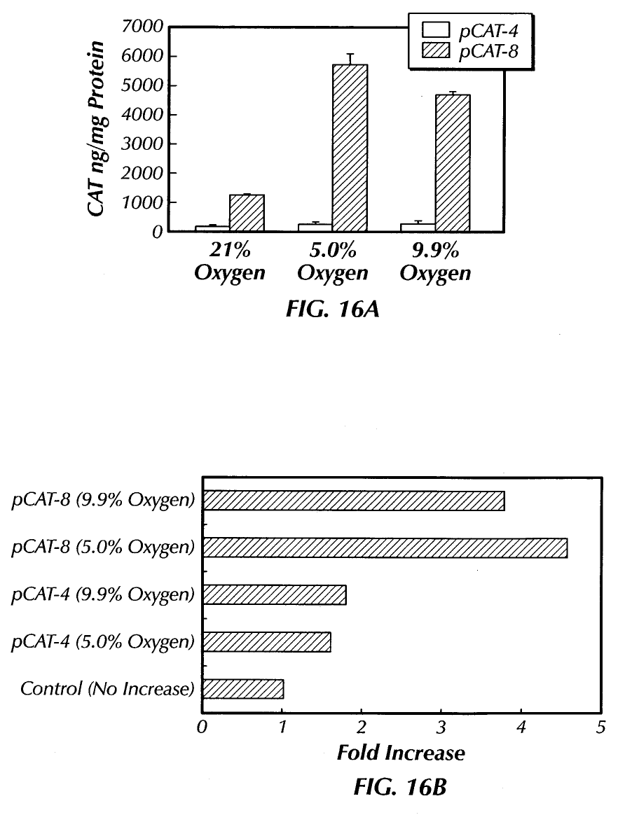

As shown in FIGS. 16A and 16B, pCAT-8 produced significantly increased levels of CAT in cells grown in 5.0% or 9.9% oxygen (p<0.01). Furthermore, MCF7 cells grown in 5.0% oxygen produced slightly higher levels of CAT than cells grown in 9.9% oxygen post-transfection with pCAT-8. No significant increase in CAT production was detected in MCF7 cells transfected with pCAT-4 and cultured in 5.0 or 9.9% oxygen post-transfection.

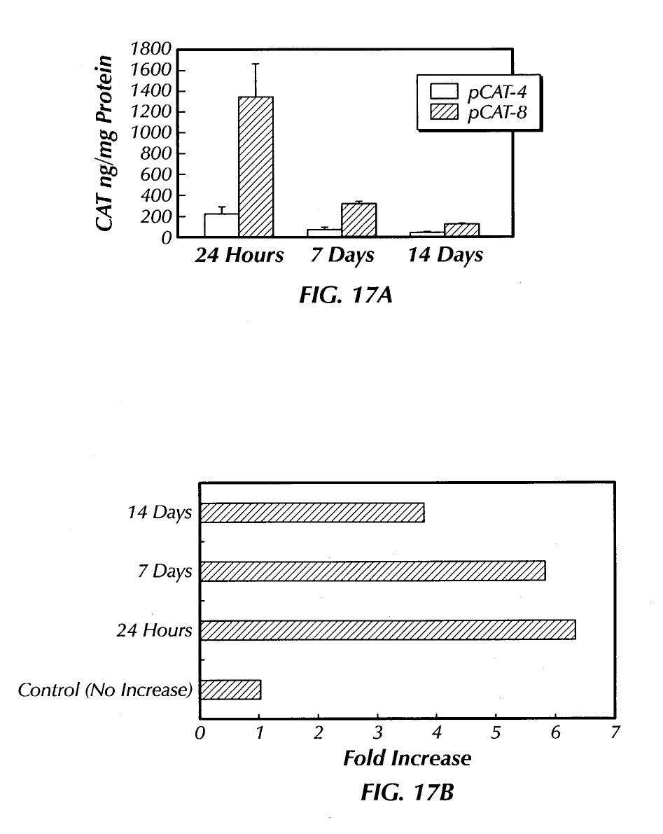

Additional experiments were performed to show that increased CAT production was not due to stabilized gene expression produced by pCAT-8. FIGS. 17A and 17B show similar declines in the levels of CAT production in MCF7 cells transfected with pCAT-4 or pCAT-8 out to 14 days post-transfection (p<0.01). Therefore, pCAT-8 produced higher levels of gene expression in MCF7 cells due to transcriptional up-regulation of the GAPDH promoter-hypoxia enhancer and to the response of the hypoxia enhancer to reduced levels of oxygen.

### EXAMPLE 7

### Gene Expression in Breast Tumors In Vivo

The transfection of breast tumors in vivo was also tested using a plasmid without the GAPDH promoter-hypoxia enhancer (i.e., the pCAT-4) and a plasmid with the GAPDH promoter-hypoxia enhancer (i.e., the pCAT-8).

A. Methodology

Mice and Mouse Tumor Model.

Human MCF7, orthotopic breast tumor xenografts were established in female, nude mice (nu/nu) implanted with estradiol tablets. Female nude mice (nu/nu), 5-6 weeks of age, were purchased from Harlan Sprague Dawley, Inc. (Indianapolis, Ind.). Each mouse was subcutaneously implanted with a 0.125 mg pellet of 17β-estradiol. The following day, MCF7 orthotopic xenografts were established in these mice by injecting 5×10⁶ MCF7 cells suspended in phosphate buffered saline. Tumors were grown to approximately 75-100 mm³.

Delivery In Vivo.

Extruded DOTAP:Chol DNA-liposome complexes were prepared as previously described (Templeton, N. S., et al. 1997). The tumor-bearing mice described above were injected with extruded DOTAP:Chol-DNA liposome complexes either by intravenous or direct tumor injections. The complexes contained either pCAT-4 or pCAT-8 plasmid DNA. For intravenous injections, 100 μl of DNA-liposome complexes containing 50 μg of DNA were slowly injected over at least a one minute period into the tail vein of the mouse using a 30-gauge syringe needle. For direct tumor injections, 100 μl of DNA-liposome complexes containing 50 μg of DNA were injected into the center of the tumor using a 30-gauge syringe needle.

Assays for CAT Production in Tissues.

Tissues were harvested and extracts prepared as previously described (Templeton, et al. 1997). Enzyme-linked immunosorbent assays (ELISAs) were performed using the Roche (Indianapolis, Ind.) CAT ELISA kit. All CAT protein determinations were corrected for any CAT immunoreactivity detected in the control tissues. Protein determinations were performed using the Micro BCA kit (Pierce, Rockford, Ill.). All experimental groups contained 10 mice per group, and controls assessed 5 mice per group. Two-sided Student's t-tests were used to determine the p-values reported. Control mice were injected with liposomes only. This work was conducted in accordance with the Baylor College of Medicine guidelines using an approved animal protocol.

B. Experimental Results

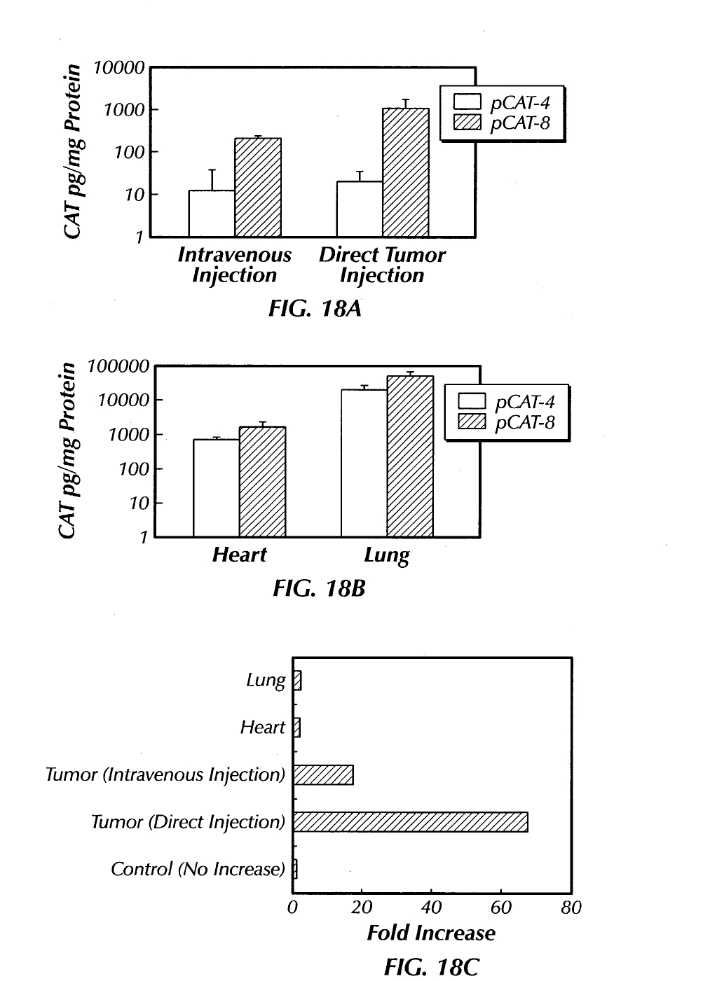

CAT production in the tumors post-injection is shown in FIG. 18A. Using pCAT-8 DNA liposomal complexes for direct tumor injections, CAT production increased 67.3-fold (FIG. 18C, p<0.025) compared to that produced by complexes containing pCAT-4 DNA. The average CAT production increased from 16 to 1076 pg CAT/mg protein in this experiment.

In addition, using pCAT-8 DNA liposomal complexes for intravenous injections, CAT production increased 16-fold (FIG. 18C, p<0.25) compared to that produced by complexes containing pCAT-4 DNA. The average CAT production after intravenous injections increased from 12 to 210 pg CAT/mg protein.

These data, along with the data shown in FIGS. 16-17, indicate that hypoxia within the breast tumor provided further increased CAT production by pCAT-8 over that produced in tissue culture transfection experiments (see FIG. 16B). Specifically, pCAT-8 increased CAT production over that of pCAT-4 by 6.2-fold in MCF7 tissue culture cells and up to 67.3-fold in MCF7 breast tumors. Since tumors are known to be hypoxic, this increased expression in tumors is thought to result from the hypoxia enhancer within the GAPDH sequences in pCAT-8. Thus, the hypoxia enhancer mediated an additional 61.1-fold increased CAT production in MCF7 breast tumors.

The heart and lungs were harvested from the identical MCF7 tumor bearing mice that had been intravenously injected with liposomal complexes containing pCAT-4 or pCAT-8 and assayed for CAT production (see FIG. 18B). The heart and lung were assayed for CAT production as previously described. Insignificant increases in CAT production, 2.2- and 2.4-fold (FIG. 18C) were observed in heart and lung tissues, respectively, using pCAT-8 versus pCAT-4 in complexes injected intravenously. Therefore, the GAPDH promoter-hypoxia enhancer specifically increased CAT production in the MCF7 tumor tissues but not in normal tissues of the same mice.

### EXAMPLE 8

### Comparisons of Gene Expression in Immune Competent, Non-Tumor Bearing Mice

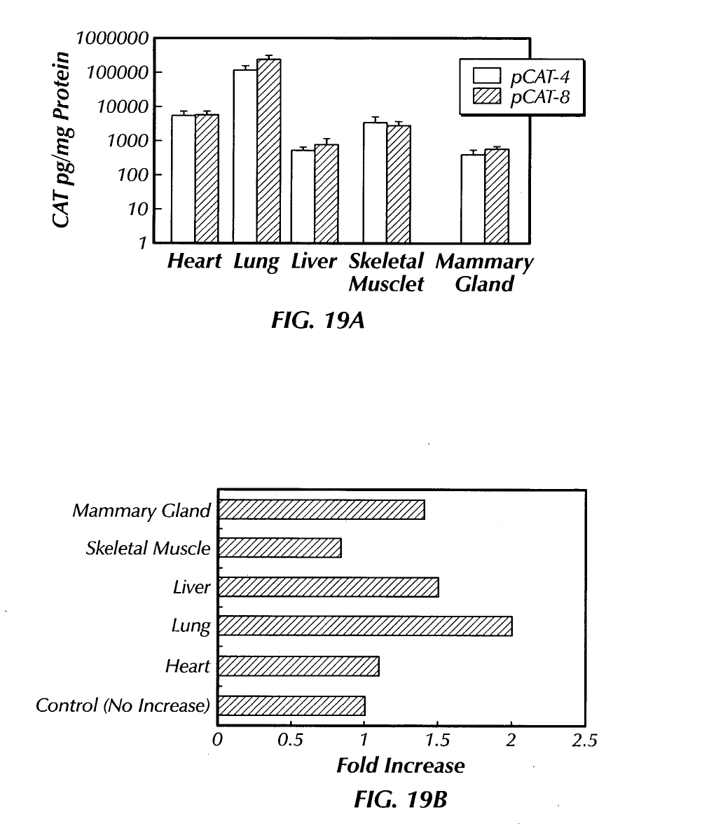

CAT production in normal, BALB/c female mice was measured after intravenous injections of extruded DOTAP:Chol DNA-liposome complexes using pCAT-8 versus pCAT-4 DNA. Female BALB/c mice, 5-6 weeks of age, were purchased from Harlan Sprague Dawley, Inc. (Indianapolis, Ind.). Heart, lungs, liver, skeletal muscle, and mammary glands were harvested and assayed for CAT production (FIG. 19A). CAT production was slightly lower in skeletal muscle using pCAT-8 versus pCAT-4 (FIG. 17B, p<0.01). Furthermore, FIG. 19B shows insignificant increases in CAT production using pCAT-8 versus pCAT-4 for heart (1.1-fold), lung (2.0-fold), liver (1.5-fold), and mammary gland (1.4-fold). Therefore, the GAPDH promoter-hypoxia enhancer did not significantly increase CAT production in normal tissues including the mammary gland, further demonstrating specific gene expression in the breast tumor (FIG. 18C) and not in normal breast tissue (FIG. 19B).

### EXAMPLE 9

### Construction of Non-viral Plasmids

The present invention sets forth a systematic approach for selecting promoters-enhancers. Presently, all plasmids used in gene therapy have a viral promoter in hopes of providing robust transcription. These viral promoters create problems because of their non-specificity and there is a need for efficient promoters that do not require the presence of a viral promoter such as the CMV promoter.

The present invention provides a systematic approach for identifying a plurality of potential promoters in a target cell. The results set out above verify the enhanced transcription seen with the use of a SAGE identified promoter. Although the data given above centers around the insertion of the GAPDH promoter-hypoxia enhancer in plasmids designed for breast tumor cells, the SAGE results identified several other promoters that were good candidates for inclusion in the plasmids. It is hypothesized that more than one of the identified promoters-enhancers can be used to provide increased production in specific cell lines.

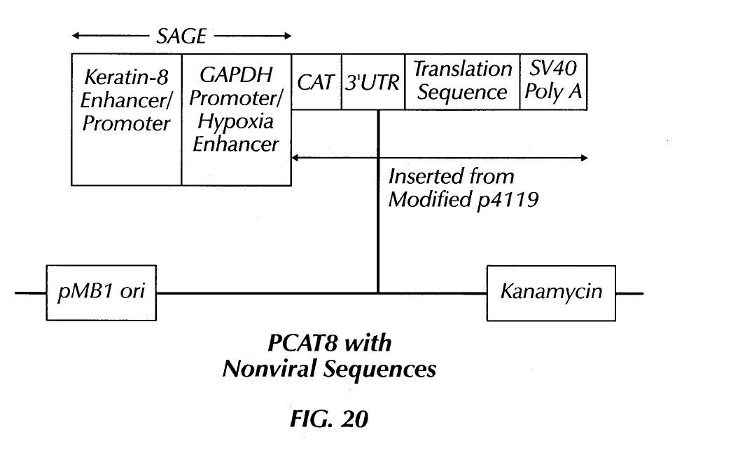

For example, in Table 1 candidate promoters-enhancers identified using the present invention include the deoythymidylate kinase promoter-enhancer, the keratin-8 promoter-enhancer, and the ribosomal L30 promoter-enhancer, as well as the GAPDH promoter-enhancer. Thus, the hypothetical plasmid shown in FIG. 20 should provide more transcription for therapeutic benefit than the GAPDH promoter-hypoxia enhancer alone, allowing sufficient transcription for therapeutic benefit without the need for the CMV promoter-enhancer or any other viral promoter. Similarly, enhancers associated with the abundant transcription products, such as keratin-8 may be used with the keratin-8 promoter or without the promoter and may be used in one or more copies.

Furthermore, other hypoxia responsive elements have been used to increase gene expression within tumors (Cao, Y. J., et al. 2001; Ruan, H., et al. 2001; Ido, A., et al. 2001; Dachs, G. U., et al. 2000; Modlich, U., et al. 2000; Shibata, T., el al. 2000). Thus, the effectiveness of the hypoxia enhancer element in increasing transcription is hypothesized to be further increased by including more than one copy of a hypoxia enhancer element in the plasmid. The hypoxia enhancer elements included may the same or different hypoxia enhancer elements.

### CONCLUSION

Production of gene expression after transfection is complex and involves more than delivery of DNA into the nucleus. Frequently, delivery of DNA into the nucleus and subsequent gene expression may be poorly correlated. Slight differences in the CMV promoter-enhancers present in plasmids produce different levels of gene expression in similar cell types. For example, FIG. 14 shows about a 4-fold difference in CAT production in MCF7 cells using the p4119 CMV promoter-enhancer (pCAT-4) versus the pVAX1 CMV promoter-enhancer (pCAT-2) for transfection in vitro. Preliminary data from transfection studies using fluorescence-labeled plasmid DNA showed no differences among levels of DNA in the nuclei, suggesting that differences in promoter efficiency rather than delivery of DNA account for the heterogeneity in expression for the eight cell lines analyzed in FIG. 14.

Several investigators have tried to use non-viral delivery systems that have demonstrated efficacy in animal models for lung cancer to treat breast tumors. These investigators have failed to show efficacy in breast tumor animal models using the same DNA expression plasmids that they have used to treat lung cancers. Their failure may well be related to their use of an inefficient delivery system and inefficient gene expression plasmids for breast tumor cells, or by the plasmid construct alone. Thus, proper plasmid design tailored for gene expression in the specific target cells is critical to making progress in gene therapy.

### REFERENCES

All patents and publications mentioned in this specification are indicative of the level of skill of those of knowledge in the art to which the invention pertains. All patents and publications referred to in this application are incorporated herein by reference to the same extent as if each was specifically indicated as being incorporated by reference and to the extent that they provide materials and methods not specifically shown.

Aki, T., Yanagisawa, S. and Akanuma, H. (1997). Identification and characterization of positive regulatory elements in the human glyceraldehyde 3-phosphate dehydrogenase gene promoter. J Biochem 122:271-278.

Baldini, N. (1997). Multidrug resistance-a multiplex phenomenon [news; comment]. Nat Med 3:378-380.

Bargou, R. C., Jurchott, K., Wagener, C., Bergmann, S., Metzner, S., Bommert, K., Mapara, M. Y., Winzer, K. J., Dietel, M., Dorken, B. and Royer H D. (1997) Nuclear localization and increased levels of transcription factor YB-1 in primary human breast cancers are associated with intrinsic BDR1 gene expression [see comments]. Nat Med 3:447-450.

Brembeck, F. H., Moffett, J., Wang, T. C., and Rustgi, A. K. (2001) Gastroenterology 120: 1720-1728.

Cao, Y. J., Shibata, T. and Rainov, N. G. (2001). Hypoxia-inducible transgene expression in differential human NT2N neurons: a cell culture model for gene therapy of postischemic neuronal loss. Gene Ther 8:1357-1362.

Dachs, G. U., Coralli, C., Hart, S. L. and Tozer, G. M. (2000). Gene delivery to hypoxic cells in vitro. Br J Cancer 83:662-667.

Del Rio, M., et al. (1999) Gene Therapy 6: 1734-1741.

Escoubet, B., Planes, C., and Clerici, C. (1999) Hypoxia increases glyceraldehyde-3-phosphate dehydrogenase transcription in rat alveolar epithelial cells. Biochem Biophys Res Commun 266(1):156-61.

Gazdar, A. F., Kurvari, V., Virmani, A., Gollahon, L., Sakaguchi, M., Westerfield, M., Kodagoda, D., Stasny, V., Cunningham, H. T., Wistuba, I. I., Tomlinson, G., Tonk, V., Ashfaq, R., Leitch, A. M., Minna, J. D. and Shay, J. W. (1998). Characterization of paired tumor and non-tumor cell lines established from patients with breast cancer. Int J Cancer 78:766-774.

Graven, K. K., Troxler, R. F., Komfeld, H., Panchenko, M. V. and Farber, H. W. (1994) Regulation of endothelial cell glyceraldehyde-3-phosphate dehydrogenase expression by hypoxia. J. Biol. Chem. 269:24446-53.

Graven, K. K., Yu, Q., Pan, D., Roncarati, J. S., and Farber, H. W. (1999) Identification of an oxygen responsive enhancer element in the glyceraldehyde-3-phosphate dehydrogenase gene. Biochim Biophys Acta 1447(2-3):208-18.

Handumrongkul, C., Zhong, W. and Debs, R. J. (2002). Distinct sets of cellular genes control the expression of transfected, nuclear-localized genes. Mol Ther 5:186-194.

Ido, A., Uto, H., Moriuchi, A., Nagata, K., Onaga, Y. and Onaga, M. (2001). Gene therapy targeting for hepatocellular carcinoma: selective and enhanced suicide gene expression regulated by a hypoxia-inducible enhancer linked to a human alpha-fetoprotein promoter. Cancer Res 61:3016-3021.

Lal, A., Lash, A. E., Altschul, S. F., Velculescu, V., Zhang, L., McLendon, R. E., Marra, M. A., Prange, C., P. J., M., Polyak, K., Papadopoulos, N., Vogelstein, B., Kinzler, K. W., Strausberg, R. L. and Riggins, G. J. (1999). A public database for gene expression in human cancers. Cancer Res 59:5403-5407.

Lash, A. E., Tolstoshev, C. M., Wagner, L., Schuler, G. D., Strausberg, R. L., Riggins, G. J. and Altschul, S. F. (2000). SAGEmap: A public gene expression resource. Genome Res 10: 1051-1060.

Liu, Y., Liggitt, D., Zhong, W., Tu, G., Gaensler, K. and Debs, R. (1995). Cationic liposome-mediated intravenous gene delivery. J Biol Chem 270:24864-24870.

Modlich, U., Pugh, C. W. and Bicknell, R. (2000). Increasing endothelial cell specific expression by the use of heterologous hypoxic and cytokine-inducible enhancers. Gene Ther 7:896-902.

Ruan, H., Su, H., Hu, L., Lamborn, K. R., Kan, Y. W. and Deen, D. F. (2001). A hypoxia-regulated adeno-associated virus vector for cancer-specific gene therapy. Neoplasia 3:255-263.

Shi, H. Y., Liang, R., Templeton, N. S. and Zhang, M. (2002). Inhibition of breast tumor progression by systemic delivery of the maspin gene in a syngeneic tumor model. Mol Ther 5:755-761.

Shibata, T., Giaccia, A. J. and Brown, J. M. (2000). Development of a hypoxia-responsive vector for tumor-specific gene therapy. Gene Ther 7:493-498.

Sinha, S., Degenstein, L., Copenhaver, C., and Fuchs, E. (2000) Mol Cell Biol 20: 2543-2555.

Sinha, S. and Fuchs, E. (2001) Proc Natl Acad Sci USA 98: 2455-2460.

Templeton, N. S., Lasic, D. D., Frederik, P. M., Strey, H. H., Roberts, D. D. and Pavlakis, G. N. (1997). Improved DNA: liposome complexes for increased systemic delivery and gene expression. Nature Biotechnology 15:647-652.

Vaupel, P. (1996). Is there a critical tissue oxygen tension for bioenergetic status and cellular pH regulation in solid tumors? Experientia 52:464-468.

Velculescu, V. E. and Zhang, L., Vogelstein, B., Kinzler, K. W. (Oct. 20, 1995). Serial analysis of gene expression. Science 270(5235):484-7.

Waikel, R. L., Kawachi, Y., Waikel, P. A., Wang, X.-J., and Roop, D. R. (2001) Nature Genetics 28: 165-168.

Yotnda, P., Chen, D.-H., Chiu, W., Piedra, P. A., Davis, A., Templeton, N. S. and Brenner, M. K. (2002). Bilamellar cationic liposomes protect adenovectors from preexisting humoral immune responses. Mol Ther 5:233-241.

Zhang, L., Zhou, W., Velculescu, V. E., Kern, S. E., Hruban, R. H., Hamilton, S. R., Vogelstein, B., and Kinzler, K. W. (May 23, 1997). Gene expression profiles in normal and cancer cells. Science 276(5316):1268-72.

Zhu, N., Liggitt, D., Liu, Y. and Debs, R. (1993). Systemic gene expression after intravenous DNA delivery in adult mice. Science 261:209-211.

While the preferred embodiment of the invention has been shown and described, modifications thereof can be made by one skilled in the art without departing from the spirit and teachings of the inventions. The embodiment described herein is exemplary only, and is not limiting. Many variations and modifications of the invention and apparatus disclosed herein are possible and are within the scope of the inventions. Accordingly, the scope of protection is not limited by the description set out above, but is only limited by the claims which follow, that scope including all equivalents of the subject matter of the claims.

What is claimed is:

1. A process for selecting a promoter for inclusion in a plasmid used to transfect a target cell, the process comprising the steps of:
identifying a transcription product in high abundance in a target cell;
identifying a promoter associated with the transcription product;
inserting the promoter into a gene expression plasmid construct, the plasmid construct having a therapeutic gene to be expressed;
transfecting the target cell with the gene expression plasmid construct; and
verifying gene expression of the therapeutic gene in the target cell.

2. The process of claim 1, wherein the transcription product is a specific mRNA.

3. The process of claim 1, wherein the transcription product is a protein.

4. The process of claim 1, wherein a Serial Analysis of Gene Expression is used to identify the transcription product.

5. The process of claim 2, wherein a SAGE database is used to identify the mRNA.

6. The process of claim 1, wherein a cDNA hybridization is used to identify the transcription product.

7. The process of claim 1, further comprising the step of identifying an enhancer associated with the promoter.

8. A process for selecting a promoter for inclusion in a plasmid to be used in gene therapy, the process comprising the steps of:
determining a gene expression level for a plurality of transcription products in a diseased tissue;
selecting a transcription product in high abundance in the diseased tissue and a target cell associated with the diseased tissue;
identifying a promoter associated with the transcription product;
inserting the promoter into a gene expression plasmid construct, the plasmid construct having a therapeutic gene to be expressed;
transfecting the target cell with the gene expression plasmid construct; and
verifying gene expression of the therapeutic gene in the target cell.

9. The process of claim 8, further comprising the steps of transfecting the diseased tissue and verifying gene expression of the therapeutic gene in the diseased tissue.

10. The process of claim 8, wherein a microarray analysis is used to identify the gene expression level for the transcription products in the diseased tissue.

11. The process of claim 8, wherein a SAGE database is used to select the transcription product.

12. The process of claim 8, further comprising the steps of identifying an enhancer associated with the promoter and inserting the enhancer into the gene expression plasmid construct.

13. The process of claim 8, wherein the diseased tissue is a breast cancer.

14. A process for designing a plasmid for transfecting a target tissue, the process comprising the steps of:
selecting a gene expression plasmid having an origin of replication, a multiple cloning site, a therapeutic gene, a polyadenylation signal sequence, and an antibiotic resistant gene;
identifying a transcription product in high abundance in a cell line associated with a target tissue;
identifying a promoter associated with the transcription product;
inserting the promoter into the gene expression plasmid in various locations close to the therapeutic gene to form a plurality of plasmid constructs;
transfecting the target cell line with each plasmid construct;
measuring gene expression of the therapeutic gene in the target cell line transfected with each plasmid construct;
selecting the plasmid constructs that provide efficient gene expression in the transfected target cell line; and
verifying gene expression of the therapeutic gene from the selected plasmid constructs in the target tissue.

15. The process of claim 14, further comprising the steps of identifying an enhancer associated with the promoter and inserting the enhancer into the gene expression plasmid.

16. The process of claim 14, wherein the antibiotic resistant gene is a Kanamycin resistant gene.

17. The process of claim 14, wherein the therapeutic gene is a p53 gene.

18. The process of claim 14, wherein the target tissue is breast tissue.

19. The process of claim 14, wherein the gene expression plasmid further comprises a constitutive promoter.

20. The process of claim 19, wherein the constitutive promoter is a viral promoter.

21. The process of claim 20, wherein the viral promoter is the cytomegalovirus promoter.

22. The process of claim 14, wherein a SAGE database is used to identify the transcription product.

23. An expression plasmid comprising:
an origin of replication gene;
a polyadenylation site;
an antibiotic resistant gene;
a multiple cloning site;
a therapeutic gene; and
a promoter selected according to the process of claim 1.

24. The expression plasmid of claim 23, wherein the antibiotic resistant gene is a Kanamycin resistant gene.

25. The expression plasmid of claim 23, wherein the therapeutic gene is a p53 gene.

26. The expression plasmid of claim 23, wherein the promoter is a glycerealdehyde 3-phosphate dehydrogenase promoter.

27. The expression plasmid of claim 23, further comprising an enhancer.

28. The expression plasmid of claim 27, wherein the enhancer is a hypoxia enhancer.

29. The expression plasmid of claim 23, further comprising more than one enhancer.

30. The expression plasmid of claim 27, wherein the expression plasmid contains more than one copy of the enhancer.

31. The expression plasmid of claim 23, further comprising a viral constitutive promoter.

32. The expression plasmid of claim 23, further comprising a CMV promoter-enhancer.

33. The expression plasmid of claim 23, wherein the expression plasmid contains more than one promoter selected according to the process of claim 1.

34. An expression plasmid for gene therapy in breast cancer, the plasmid comprising:
an origin of replication gene;
a polyadenylation site;
an antibiotic resistant gene;
a multiple cloning site;
a therapeutic gene;
a GADPH promoter; and
a hypoxia enhancer.

35. The plasmid of claim 34, further comprising a keratin-8 promoter-enhancer.

36. The plasmid of claim 34 having multiple copies of the hypoxia enhancer.

37. The plasmid of claim 34, wherein the GAPDH promoter and the hypoxia enhancer are situated 5′ of the therapeutic gene.

38. The plasmid of claim 37, wherein the GAPDH promoter and the hypoxia enhancer are adjacent the therapeutic gene.
